(19) United States
(12) Patent Application Publication
(10) Pub. No.: US 2023/0047769 A1
(43) Pub. Date: Feb. 16, 2023

(54) BIOLOGICAL SUBSTANCE DETECTION CHIP, BIOLOGICAL SUBSTANCE DETECTION DEVICE AND BIOLOGICAL SUBSTANCE DETECTION SYSTEM

(71) Applicant: SONY SEMICONDUCTOR SOLUTIONS CORPORATION, KANAGAWA (JP)

(72) Inventors: HARUMI TANAKA, TOKYO (JP); YOSHIAKI MASUDA, KANAGAWA (JP)

(21) Appl. No.: 17/904,177

(22) PCT Filed: Jan. 29, 2021
(86) PCT No.: PCT/JP2021/003235

§ 371 Date: Aug. 12, 2022

(30) Foreign Application Priority Data
Feb. 19, 2020 (JP) 2020-026051

(51) Int. Cl. B01L 3/00; G01N 21/64

(52) U.S. Cl. CPC B01L 3/502761; G01N 21/6428; B01L 2300/0636; B01L 2300/0645; B01L 2200/0652; B01L 2200/16; G01N 2021/6439

(57) ABSTRACT

There is provided a biological substance detection chip having high detection accuracy. The present technology provides a biological substance detection chip which is composed of a plurality of pixels in which the pixel includes at least a holding surface on which a biological substance is held and a photoelectric conversion unit that is provided below the holding surface and provided on a semiconductor substrate, wherein a partition wall made of a conductor is provided between the pixels on the holding surface. In addition, the present technology provides a biological substance detection device and a biological substance detection system using the biological substance detection chip.

### TECHNICAL FIELD

The present technology relates to a biological substance detection chip, a biological substance detection device and a biological substance detection system.

### BACKGROUND ART

In recent years, technical research on gene analysis, protein analysis, cell analysis and the like has progressed in various fields such as medicine, drug discovery, clinical examination, food, agriculture, and engineering. In particular, recently, the development and practical application of detection technology on chips such as lab-on-a-chip in which various reactions such as detection and analysis of biological substances such as nucleic acids, proteins, cells, and microorganisms are performed in microscale channels and wells provided in the chips have been progressed. These are being focused on as a method of easily measuring biological substances and the like.

For example, PTL 1 discloses an optical detection device including at least a first substrate in which a plurality of wells are formed, a second substrate in which a heating unit is provided so that it comes in contact with the wells, a third substrate in which a plurality of light emitting units are positioned to correspond to the positions of the wells, and a fourth substrate in which a plurality of light detecting units are positioned to correspond to the positions of the wells. In this optical detection device, various reactions that proceed in the wells can be measured.

In addition, for example, PTL 2 discloses a chemical sensor including a substrate in which an optical detection unit is formed, and a plasmon absorption layer laminated on the substrate and having a metal nanostructure that causes plasmon absorption. This chemical sensor can detect emission of light caused by binding between a probe material fixed on the sensor and a target material.

### CITATION LIST

### Patent Literature

### [PTL 1]

JP 2010-284152A

### [PTL 2]

WO 2013/080473

### SUMMARY

### Technical Problem

Even if proteins such as DNA and antibodies, and biological substances such as cells are suspended in a sample liquid or fixed, they may shrink due to their higher-order structure, which may influence light detection.

Therefore, a main object of the present technology is to provide a biological substance detection chip having high detection accuracy.

### Solution to Problem

Specifically, first, the present technology provides a biological substance detection chip which is composed of a plurality of pixels in which the pixel at least includes a holding surface on which a biological substance is held and a photoelectric conversion unit that is provided below the holding surface and provided on a semiconductor substrate, wherein a partition wall made of a conductor is provided between the pixels on the holding surface.

In the biological substance detection chip according to the present technology, the partition wall can be designed so that a voltage is applied when the biological substance is detected.

In this case, a positive voltage or a negative voltage can be applied to all of the partition walls, and for the partition walls, a positive voltage or a negative voltage can be applied to the respective partition walls.

In addition, a magnitude of a voltage applied to the partition walls may be changed for each partition wall.

In the biological substance detection chip according to the present technology, some or all of the partition walls that are able to be conductive on the holding surface may be covered with a protective film.

Regarding the biological substance that can be detected by the biological substance detection chip according to the present technology, one or more biological substances selected from among nucleic acids, proteins, cells, microorganisms, chromosomes, ribosomes, mitochondria, organelles (cell organelles), and complexes thereof may be exemplified.

Next, the present technology provides a biological substance detection device, including a biological substance detection chip which is composed of a plurality of pixels, in which the pixel includes at least a holding surface on which a biological substance is held and a photoelectric conversion unit that is provided below the holding surface, and in which a partition wall made of a conductor is provided between the pixels on the holding surface, and an analysis unit that analyzes electrical information acquired by the biological substance detection chip.

The present technology also provides a biological substance detection system, including a biological substance detection chip which is composed of a plurality of pixels, in which the pixel includes at least a holding surface on which a biological substance is held and a photoelectric conversion unit that is provided below the holding surface, and in which a partition wall made of a conductor is provided between the pixels on the holding surface; and an analysis device that analyzes electrical information acquired by the biological substance detection chip.

In the present technology, “biological substance” widely includes nucleic acids, proteins, cells, microorganisms, chromosomes, ribosomes, mitochondria, organelles (cell organelles), complexes thereof, and the like. Cells include animal cells (such as blood cell lineage cells) and plant cells. Microorganisms include bacteria such as E. coli, viruses such as tobacco mosaic virus, and fungi such as yeast.

### BRIEF DESCRIPTION OF DRAWINGS

FIG. 1 is a schematic conceptual view schematically showing interactions between biological substances S that can be detected by a biological substance detection chip 1, a biological substance detection device 2, and a biological substance detection system 3 according to the present technology.

FIG. 2 is a schematic conceptual view schematically showing interactions between biological substances S that can be detected by the biological substance detection chip 1, the biological substance detection device 2, and the biological substance detection system 3 according to the present technology.

FIG. 3 is a schematic conceptual view schematically showing interactions between biological substances S that can be detected by the biological substance detection chip 1, the biological substance detection device 2, and the biological substance detection system 3 according to the present technology.

FIG. 4 is a schematic conceptual view schematically showing screening of other substances that can be performed by the biological substance detection chip 1, the biological substance detection device 2, and the biological substance detection system 3 according to the present technology.

FIG. 5 is a schematic conceptual view schematically showing screening of other substances that can be performed by the biological substance detection chip 1, the biological substance detection device 2, and the biological substance detection system 3 according to the present technology.

FIG. 6 is a schematic conceptual view schematically showing screening of other substances that can be performed by the biological substance detection chip 1, the biological substance detection device 2, and the biological substance detection system 3 according to the present technology.

FIG. 7 is a schematic plan view schematically showing a first embodiment of the biological substance detection chip 1 according to the present technology as viewed from above.

FIG. 8 is a schematic end view taken along the line A-A, schematically showing the first embodiment of the biological substance detection chip 1 according to the present technology.

FIG. 9 is a schematic end view schematically showing a modified example of the first embodiment of the biological substance detection chip 1 according to the present technology.

FIG. 10 is a schematic plan view schematically showing a second embodiment of the biological substance detection chip 1 according to the present technology as viewed from above.

FIG. 11 is a schematic plan view schematically showing a first modified example of the second embodiment of the biological substance detection chip 1 according to the present technology as viewed from above.

FIG. 12 is a schematic plan view schematically showing a second modified example of the second embodiment of the biological substance detection chip 1 according to the present technology as viewed from above.

FIG. 13 is a schematic plan view schematically showing a third modified example of the second embodiment of the biological substance detection chip 1 according to the present technology as viewed from above.

FIG. 14 is a schematic plan view schematically showing a third embodiment of the biological substance detection chip 1 according to the present technology as viewed from above.

FIG. 15 is a schematic plan view schematically showing a first modified example of the third embodiment of the biological substance detection chip 1 according to the present technology as viewed from above.

FIG. 16 is a schematic plan view schematically showing a second modified example of the third embodiment of the biological substance detection chip 1 according to the present technology as viewed from above.

FIG. 17 is a schematic plan view schematically showing a third modified example of the third embodiment of the biological substance detection chip 1 according to the present technology as viewed from above.

FIG. 18 is a schematic plan view schematically showing a fourth embodiment of the biological substance detection chip 1 according to the present technology as viewed from above.

FIG. 19 is a schematic plan view schematically showing a modified example of the fourth embodiment of the biological substance detection chip 1 according to the present technology as viewed from above.

FIG. 20 is a schematic end view taken along the line B-B, schematically showing the first embodiment of the biological substance detection chip 1 according to the present technology.

FIG. 21 is a schematic end view taken along the line C-C, schematically showing the third embodiment of the biological substance detection chip 1 according to the present technology.

FIG. 22 is a block diagram showing a concept of the biological substance detection device 2 according to the present technology.

FIG. 23 is a block diagram showing a concept of the biological substance detection system 3 according to the present technology.

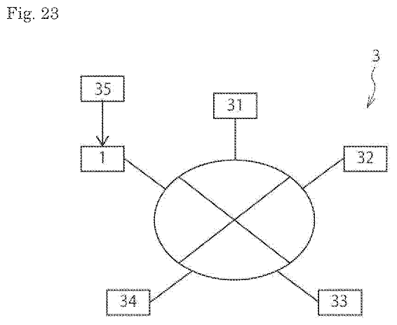

### Description of Embodiments

Hereinafter, preferable embodiments for implementing the present technology will be described with reference to the drawings. The embodiments described below show examples of representative embodiments of the present technology, but the scope of the present technology should not be narrowly understood based on the embodiments. Here, description will proceed in the following order.

1. Overview of Biological Substance Detection performed by Present Technology

(1) Detection of Biological Substance S Itself

(2) Detection of Interactions of Biological Substance S

(3) Screening of Other Substances

2. Biological Substance Detection Chip 1

(1) First Embodiment

(2) Second Embodiment

(3) Third Embodiment

(4) Fourth Embodiment

(5) Other Examples

(6) Method of Applying Voltage

3. Biological Substance Detection Device 2

4. Biological Substance Detection System 3

### 1. Overview of Biological Substance Detection Performed by Present Technology

An overview of detection of a biological substance S performed by a biological substance detection chip 1, a biological substance detection device 2, and a biological substance detection system 3 according to the present technology will be described. The biological substance detection chip 1 and the biological substance detection device 2, and the biological substance detection system 3 according to the present technology can be used for (1) detection of a biological substance S itself, (2) detection of interactions of the biological substance S, (3) screening of other substances (for example, medicinal components) using biological substance S and the like. Here, each detection is performed on a holding surface 111 of the biological substance detection chip 1 to be described below.

### (1) Detection of Biological Substance S Itself

For example, the present technology can be used for detecting bio-substances such as red blood cells, white blood cells, platelets, cytokines, hormone substances, sugars, lipids, proteins and the like contained in body fluids such as blood, urine, feces, and saliva; microorganisms such as bacteria, fungi, viruses and the like contained in body fluids and water; and genes in cells and microorganisms. For example, after staining with a dye that acts specifically on a detection target substance or a non-detection target substance, the presence of a detection target substance can be detected depending on the presence of desired light detection. The detection results can be used for disease diagnosis, internal environment diagnosis, water quality examination and the like.

### (2) Detection of Interactions of Biological Substance S

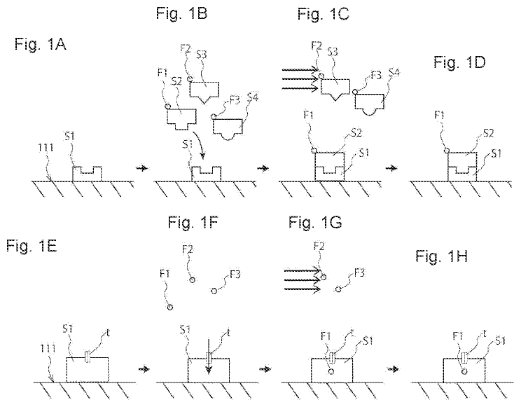

For example, the present technology can be used to detect interactions such as protein interactions, nucleic acid hybridization, and binding of cytokines and hormone substances to receptors. Specific detection examples will be described with reference to FIGS. 1 to 3.

For example, as shown in A to D in FIG. 1, a biological substance 51 such as a protein or a receptor (or an imitation of a receptor) is fixed on a holding surface 111 (refer to A in FIG. 1), and fixed dyes such as fluorescent dyes F1 to F3 are added to biological substances S2 to S4 for checking the interaction thereof (refer to B in FIG. 1). Then, the biological substances S3 and S4 that do not interact with the biological substance S1 are washed off (refer to C in FIG. 1), and the interaction between the biological substance S1 and the biological substance S2 can be detected by detecting the fluorescent dye F1 from the holding surface 111 (refer to D in FIG. 1).

For example, as shown in E to H in FIG. 1, the biological substance S1 such as a cell is fixed on the holding surface 111, and an entrapped illuminant F1 can be detected via a transporter t (for example, a transporter in a cell membrane) of the biological substance S1.

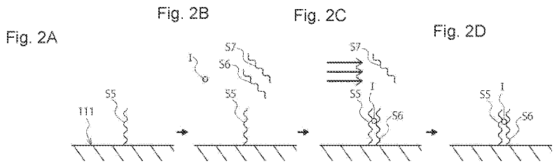

For example, as shown in A to D in FIG. 2, a probe S5 composed of DNA, RNA or the like is fixed to the holding surface 111 (refer to A in FIG. 2), and a sample containing DNA S6 and S7 that can be targets, and an intercalator I are added (refer to B in FIG. 2). Then, when the DNA S6 having a sequence complementary to the probe S5 is contained in the sample, a hybridization reaction occurs. The DNA S7 that is not hybridized is washed off (refer to C in FIG. 2), and hybridization between the probe S5 and the target DNA S6 can be detected by detecting light from the intercalator I from the holding surface 111 (refer to D in FIG. 2).

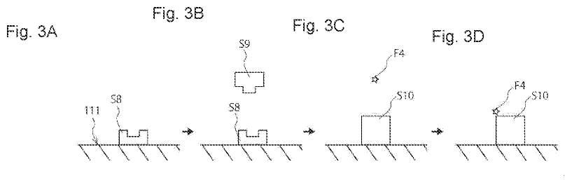

For example, as shown in A to D in FIG. 3, a biological substance S8 is fixed on the holding surface 111(refer to A in FIG. 3), and a biological substance S9 that interacts with the biological substance S8 to form a new substance S10 is added (refer to B in FIG. 3). Next, a dye such as a fluorescent dye F4 that specifically binds to the substance S10 is added (refer to C in FIG. 3), and the fluorescent dye F4 is detected from the holding surface 111 (refer to D in FIG. 3), and thus the interaction between the biological substance S8 and the biological substance S9 can be detected.

### (3) Screening of Other Substances

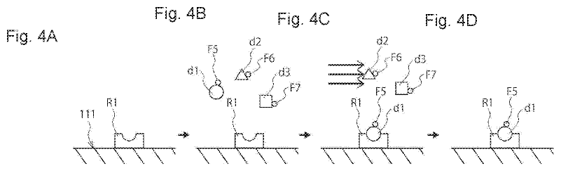

For example, the present technology can be used for screening of substances that can be agonists or antagonists of various receptors, and screening of agents for inhibiting production of various microorganisms, antibacterial agents, bactericidal agents and the like. Specific detection examples will be described with reference to FIG. 4 to FIG. 6.

For example, as shown in A to D in FIG. 4, a receptor R1 (or an imitation of the receptor R1) is fixed on the holding surface 111 (refer to A in FIG. 4), and fixed dyes such as fluorescent dyes F5 to F7 are added to substances d1 to d3 for checking operability of the receptor R1 (refer to B in FIG. 4). Then, the substances d2 and d3 that do not bind to the receptor R1 are washed off (refer to C in FIG. 4), and it is possible to perform screening of the substance d1 that can be an agonist of the receptor R1 by detecting the fluorescent dye F5 from the holding surface 111 (refer to D in FIG. 3).

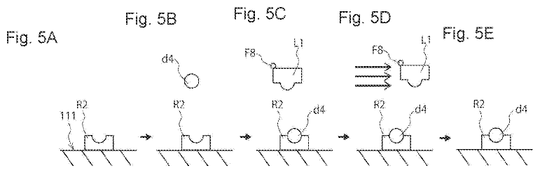

For example, as shown in A to E in FIG. 5, a receptor R2 (or an imitation of the receptor R2) is fixed on the holding surface 111 (refer to A in FIG. 5), and a substance d4 for checking antagonism of the receptor R2 is added (refer to B in FIG. 5). Next, a ligand L1 that binds to the receptor R2 to which a dye such as a fluorescent dye F8 is fixed is added (refer to C in FIG. 5). In this case, if the substance d4 can be an antagonist of the receptor R2, the ligand L1 cannot bind to the receptor R2 because the receptor R2 and the substance d4 are already bound to each other (refer to C in FIG. 5). In this state, after the ligand L1 that does not bind to the receptor R2 is washed off (refer to D in FIG. 5), even if an attempt is made to detect the fluorescent dye F8 from the holding surface 111, light is not detected because the fluorescent dye F8 is not present on the holding surface 111 due to the washing (refer to E in FIG. 5).

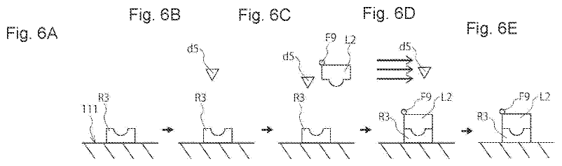

On the other hand, for example, as shown in A to E in FIG. 6, a receptor R3 (or an imitation of the receptor R3) is fixed on the holding surface 111 (refer to A in FIG. 6), and a substance d5 for checking antagonism of the receptor R3 is added (refer to B in FIG. 6). Next, a ligand L2 that binds to the receptor R3 to which a dye such as a fluorescent dye F9 is fixed is added (refer to C in FIG. 6). In this case, when the substance d5 cannot be an antagonist of the receptor R3, the ligand L2 binds to the receptor R3 (refer to D in FIG. 6). In this state, when the substance d5 that does not bind to the receptor R3 is washed off (refer to D in FIG. 6), the fluorescent dye F9 is detected from the holding surface 111 (refer to E in FIG. 6).

In this manner, as shown in FIG. 5 and FIG. 6, it is possible to perform screening of the substance d4 that can be an antagonist of the receptor R3 depending on whether the fluorescent dye F8 or the fluorescent dye F9 is detected from the holding surface 111.

### 2. Biological Substance Detection Chip 1

The biological substance detection chip 1 according to the present technology is composed of a plurality of pixels 11, and the pixel 11 includes at least a holding surface 111 on which a biological substance S is held and a photoelectric conversion unit 112 that is provided below the holding surface 111 and provided on a semiconductor substrate 12. Here, a partition wall 13 made of a conductor is provided between the pixels 11 on the holding surface 111. Hereinafter, description will proceed with reference to embodiments.

Examples of conductors constituting the partition wall 13 include a metal, and regarding the metal, for example, tungsten (W), aluminum (Al), copper (Cu), titanium (Ti) or the like can be used.

### (1) FIRST EMBODIMENT

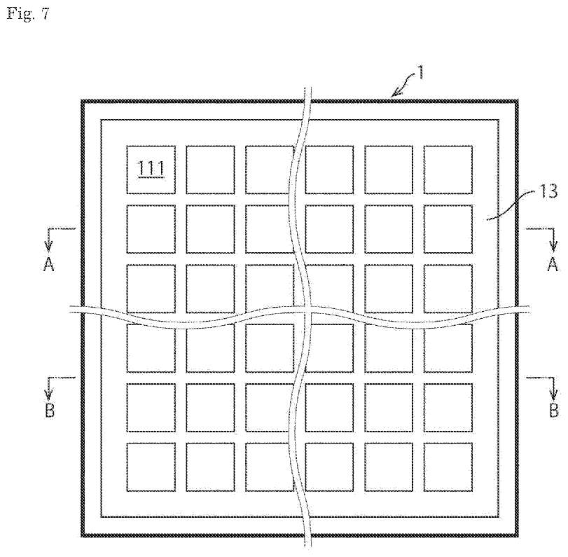

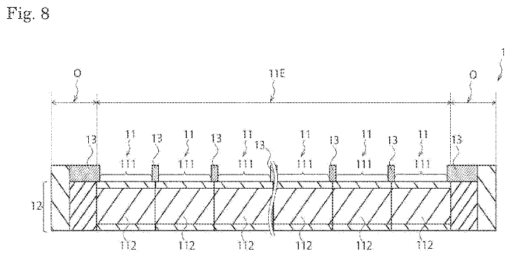

FIG. 7 is a schematic plan view schematically showing a first embodiment of the biological substance detection chip 1 according to the present technology as viewed from above, and FIG. 8 is a schematic end view taken along the line A-A, schematically showing the first embodiment of the biological substance detection chip 1 according to the present technology. The biological substance detection chip 1 according to the first embodiment has an effective pixel region 11E in which a plurality of pixels 11 are two-dimensionally arranged in a matrix. Each pixel 11 includes at least a holding surface 111 on which a biological substance S is held and a photoelectric conversion unit 112. In the photoelectric conversion unit 112, for example, a photoelectric conversion element such as a photodiode can be freely used. In addition, although not shown, each pixel 11 may include a pixel circuit composed of a charge storage unit, a plurality of transistors, a capacitive element and the like. Although not shown, an optical black pixel, a wiring region and the like can be provided on the outside (invalid pixel region O) of the effective pixel region 11E.

The holding surface 111 is not particularly limited as long as it has a configuration that can hold the biological substance S, and a surface treatment can be freely used. For example, the holding surface 111 can be formed by applying a photosensitive silane coupling agent or the like that is modified with ultraviolet ray emission to be hydrophilic and selectively emitting ultraviolet rays to a region in which the biological substance S is desired to be held. In addition, for example, when the holding surface 111 is treated with avidin, the biological substance S such as a nucleic acid whose one end is biotinylated can be held by an avidin-biotin bond. In addition, according to the configuration in which a liquid can be held on the holding surface 111, it is also possible to hold the biological substance S in the liquid.

Since the partition wall 13 is made of a conductor, a voltage can be applied. For example, if a voltage is applied to the partition wall 13 when the biological substance S is held on the holding surface 111, the partition wall 13 functions as an electrode for attracting the charged biological substance S, and pushing it to a desired location such as the center of a pixel.

The specific structure of the partition wall 13 is not particularly limited as long as it is provided between the pixels 11 on the holding surface 111. For example, as shown in FIG. 8, each pixel 11 can be completely partitioned by the partition wall 13. In this case, for example, when a positive voltage or a negative voltage is applied to all of the partition walls 13 according to the positive and negative charge of the biological substance S held on the holding surface 111, the biological substance S can be attracted to the partition wall 13 or collected in the center of the pixel. More specifically, for example, when negatively charged DNA is detected, if a negative voltage is applied to all of the partition walls 13, DNA can be collected in the center of the pixel. As a result, it is possible to improve detection accuracy.

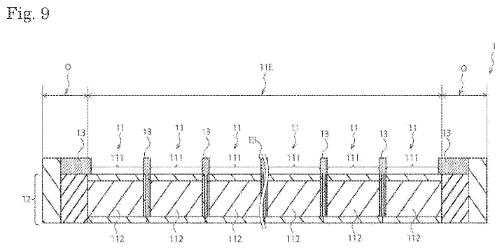

Here, as shown in the schematic cross-sectional view schematically showing a modified example of the first embodiment of the biological substance detection chip 1 according to the present technology of FIG. 9, the partition wall 13 can have a configuration in which it is embedded in the semiconductor substrate 12. When the partition wall 13 is embedded in the semiconductor substrate 12, it is possible to prevent light from leaking between pixels, and it is possible to further improve detection accuracy.

### (2) SECOND EMBODIMENT

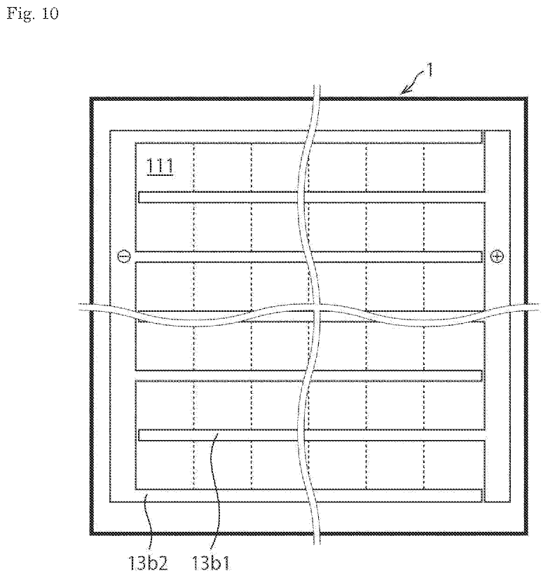

FIG. 10 is a schematic plan view schematically showing a second embodiment of the biological substance detection chip 1 according to the present technology as viewed from above. The biological substance detection chip 1 according to the second embodiment is an example in which the partition wall 13 is not present in the vertical direction as viewed from above, and the partition wall 13 is present only in the lateral direction. In this case, for example, when a positive voltage or a negative voltage is alternately applied to the partition wall 13 in the lateral direction as viewed from above, the orientation of the biological substance S can be aligned in a desired direction. More specifically, for example, when negatively charged DNA is detected, as in the second embodiment shown in FIG. 10, if a positive voltage or a negative voltage is alternately applied to the partition wall 13 in the lateral direction as viewed from above, the orientation of DNA can be aligned. As a result, it is possible to improve detection accuracy.

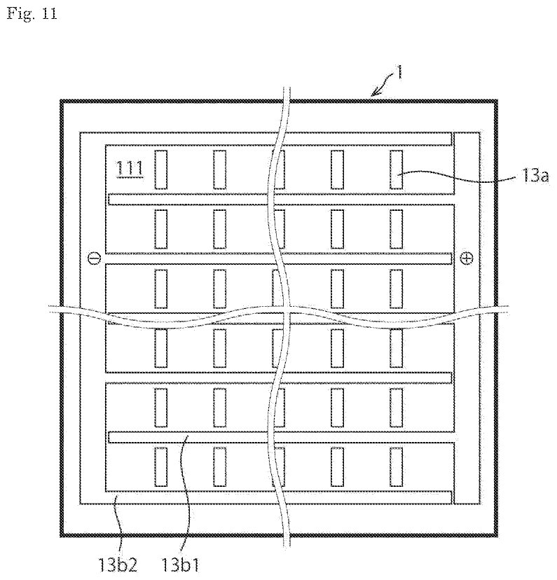

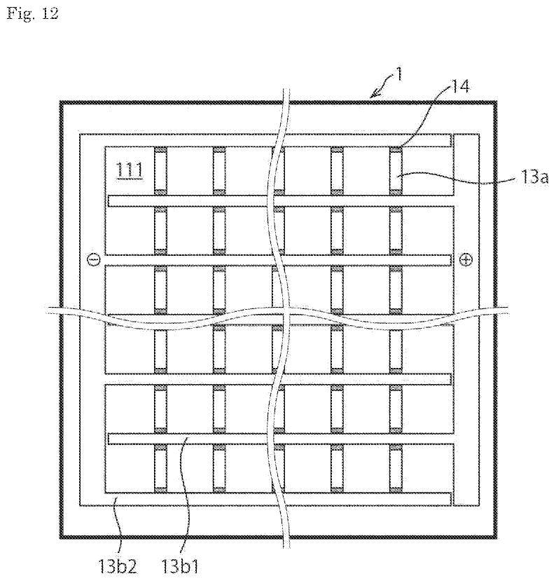

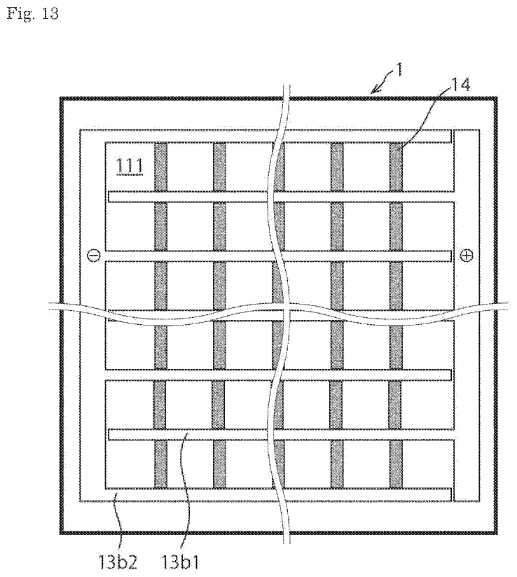

Here, for example, as shown in the schematic cross-sectional view schematically showing a first modified example of the second embodiment of the biological substance detection chip 1 according to the present technology of FIG. 11, in order to divide a voltage, a partition wall 13a in the vertical direction as viewed from above can be provided with a space between it and a partition wall 13b in the lateral direction as viewed from above. In this case, for example, as shown in the schematic cross-sectional view schematically showing a second modified example of the second embodiment of the biological substance detection chip 1 according to the present technology of FIG. 12, an insulator 14 may be provided between the partition wall 13a in the vertical direction as viewed from above and the partition wall 13b in the lateral direction as viewed from above. In addition, for example, as shown in the schematic cross-sectional view schematically showing a third modified example of the second embodiment of the biological substance detection chip 1 according to the present technology of FIG. 13, a partition wall made of the insulator 14 can be provided in the vertical direction as viewed from above.

An insulating material that can be used for the biological substance detection chip 1 can be used as the insulator 14 as long as the effects of the present technology are not impaired. For example, an oxide film of silicon oxide (SiO₂) or the like, and a nitride film of silicon nitride (Si₃N₄), silicon oxynitride (SiON) or the like can be used.

### (3) THIRD EMBODIMENT

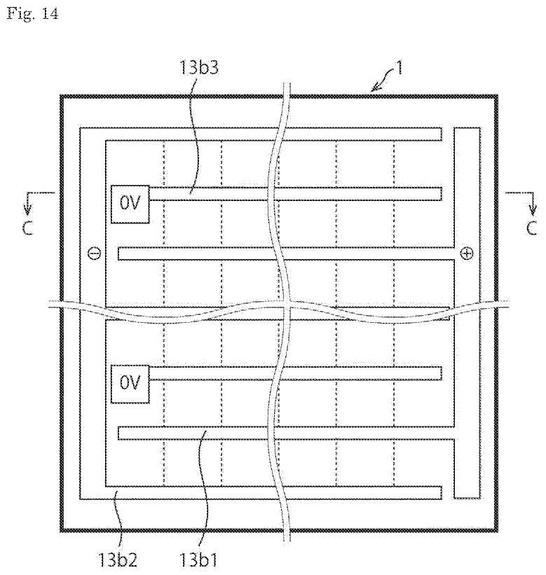

FIG. 14 is a schematic plan view schematically showing a third embodiment of the biological substance detection chip 1 according to the present technology as viewed from above. The biological substance detection chip 1 according to the third embodiment is an example in which the partition wall 13 is not present in the vertical direction as viewed from above, and the partition wall 13 is present only in the lateral direction. In addition, this is an example in which a 0 V partition wall 13b3 is arranged between a partition wall 13b1 to which a positive voltage is applied and a partition wall 13b2 to which a negative voltage is applied. When the 0 V partition wall 13b3 is arranged, it is possible to stabilize the charge and form a flow of the biological substance S. More specifically, for example, when negatively charged DNA is detected, as in the third embodiment shown in FIG. 14, when the partition wall 13b2 to which a negative voltage is applied, the 0 V partition wall 13b3, and the partition wall 13b1 to which a positive voltage is applied are arranged in that order, DNA can flow from the − side to the + side. For example, DNA can be separated because the flow may differ depending on the difference in the charge of DNA. As a result, detection accuracy can be improved and additional information can be obtained.

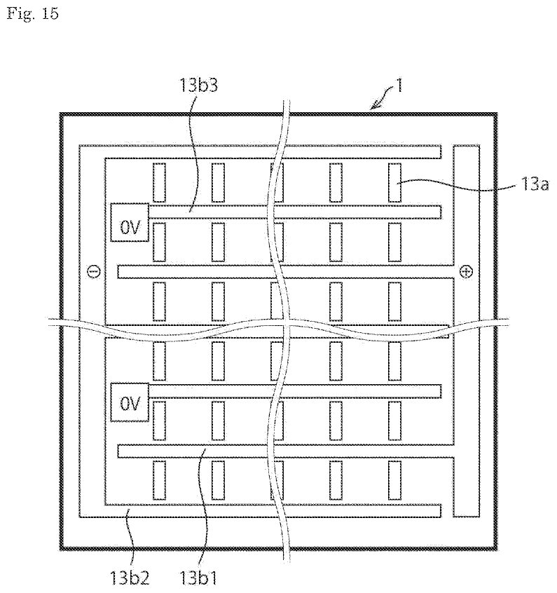

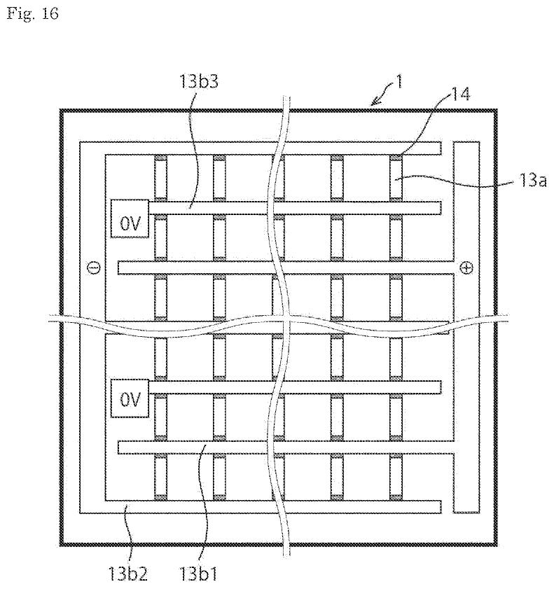

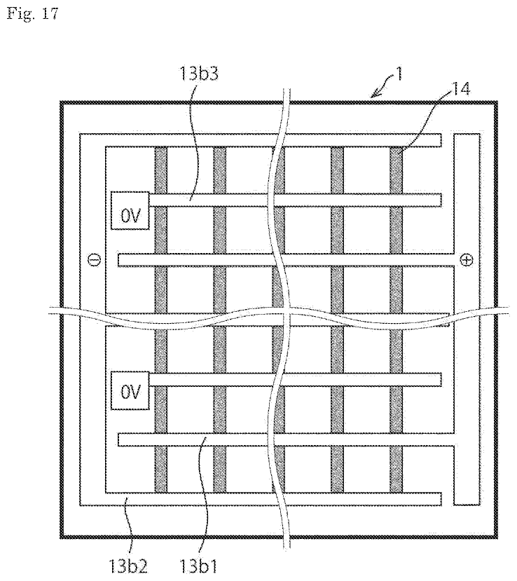

Here, as in the second embodiment, for example, as shown in the schematic cross-sectional view schematically showing a first modified example of the third embodiment of the biological substance detection chip 1 according to the present technology of FIG. 15, in order to divide a voltage, the partition wall 13a in the vertical direction as viewed from above can be provided with a space between it and the partition walls 13b1 to 3 in the lateral direction as viewed from above. In this case, for example, as shown in the schematic cross-sectional view schematically showing a second modified example of the third embodiment of the biological substance detection chip 1 according to the present technology of FIG. 16, the insulator 14 may be provided between the partition wall 13a in the vertical direction as viewed from above and the partition walls 13b1 to 3 in the lateral direction as viewed from above. In addition, for example, as shown in the schematic cross-sectional view schematically showing a third modified example of the third embodiment of the biological substance detection chip 1 according to the present technology of FIG. 17, a partition wall made of the insulator 14 can be provided in the vertical direction as viewed from above.

### (4) FOURTH EMBODIMENT

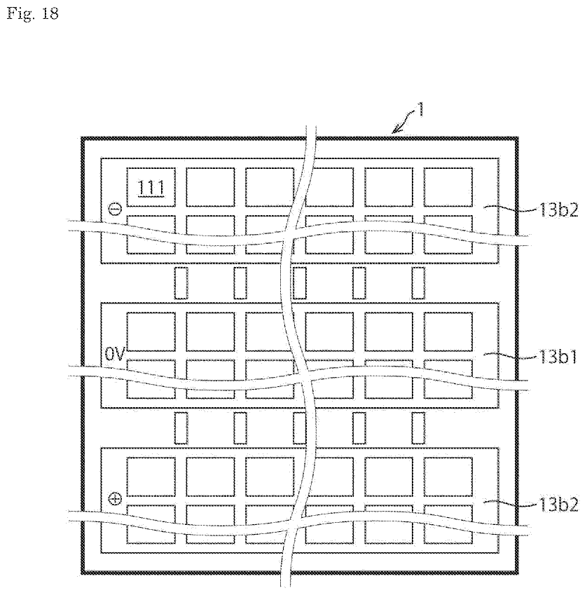

FIG. 18 is a schematic plan view schematically showing a fourth embodiment of the biological substance detection chip 1 according to the present technology as viewed from above. The biological substance detection chip 1 according to the fourth embodiment is an example including three regions: a region of the partition wall 13b1 to which a positive voltage is applied, a region of the partition wall 13b2 to which a negative voltage is applied, and a region of the 0 V partition wall 13b3. When the region of the 0 V partition wall 13b3 is provided, it is possible to stabilize the charge and form a flow of the biological substance S. More specifically, for example, when negatively charged DNA is detected, as in the fourth embodiment shown in FIG. 18, when the partition wall 13b2 to which a negative voltage is applied, the 0 V partition wall 13b3, and the partition wall 13b1 to which a positive voltage is applied are arranged in that order, DNA can flow from the − side to the + side. For example, DNA can be separated because the flow may differ depending on the difference in the charge of DNA. As a result, detection accuracy can be improved and additional information can be obtained.

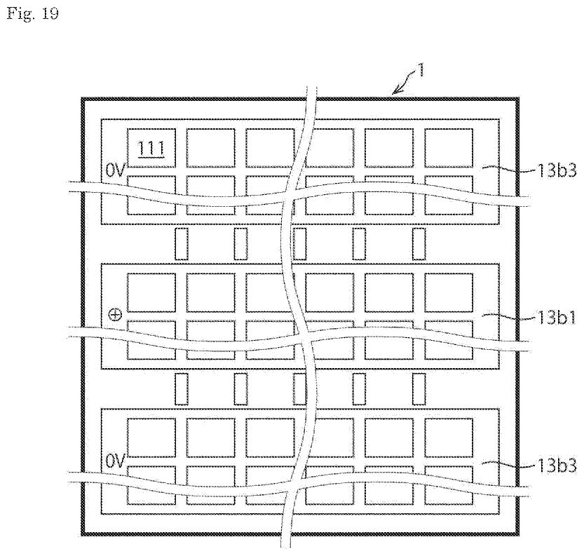

FIG. 19 is a schematic plan view schematically showing a modified example of the fourth embodiment of the biological substance detection chip 1 according to the present technology as viewed from above. As in this modified example, when the region of the partition walls 13b2 and 13b1 to which a positive voltage or a negative voltage is applied is inserted between the regions of the 0 V partition walls 13b3, the biological substance S can be collected in the center of the biological substance detection chip 1. More specifically, for example, when negatively charged DNA is detected, as in the modified example of the fourth embodiment shown in FIG. 19, if the region of the partition wall 13b1 to which a positive voltage is applied is inserted between the regions of the 0 V partition walls 13b3, DNA can be collected in the center of the biological substance detection chip 1.

### (5) OTHER EXAMPLES

As other examples, although not shown, for example, the magnitude of the voltage applied to the partition wall 13 can be adjusted. For example, for each area, when the magnitude of the voltage applied to the partition wall 13 is adjusted, it is possible to collect a desired biological substance S for each area according to the charge of the biological substance S.

Although not shown, some or all of the partition walls 13 described above may be covered with a protective film. When the partition wall 13 is covered with a protective film, the thinness of the protective film and the material of the protective film are selected so that the partition wall 13 is conductive on the holding surface 111. When the protective film is provided, weather resistance to heat, light, water, acids, alkalis, chemicals and the like can be improved, and it is possible to keep the partition wall 13 in contact with water, acid, alkalis, or chemicals for a long time.

The material forming the protective film can be freely selected as long as the effects of the present technology are not impaired. For example, silicon oxide (SiO₂), silicon nitride (Si₃N₄), silicon oxynitride (SiON) and the like can be used.

### (6) Method of Applying Voltage

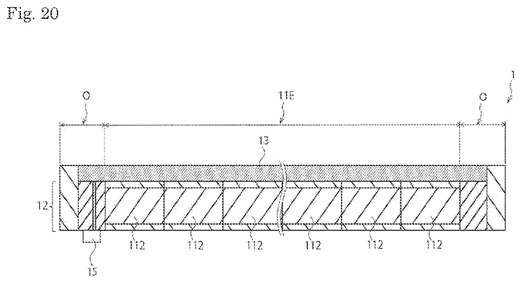

The method of applying a voltage to the partition wall 13 can be freely designed as long as the effects of the present technology are not impaired. For example, as shown in the end view taken along the line B-B, schematically showing the first embodiment of the biological substance detection chip 1 according to the present technology shown in FIG. 20, in the invalid pixel region O, a voltage can be applied by connecting the partition wall 13 to a gate 15 via the semiconductor substrate 12. In this case, the gate 15 can control the positive charge or the negative charge. In addition, although not shown, in the partition wall 13 of the invalid pixel region O, it is possible to apply an external voltage from the upper side of the chip.

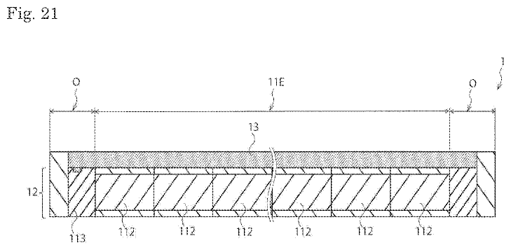

The method in which the partition wall 13 has 0 V can be freely designed as long as the effects of the present technology are not impaired. For example, as shown in the schematic end view taken along the line C-C, schematically showing the third embodiment of the biological substance detection chip 1 according to the present technology shown in FIG. 21, 0 V can also be obtained by connecting the partition wall 13b3 to a P-type region 113.

### 3. Biological Substance Detection Device 2

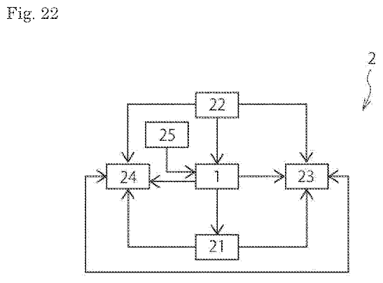

FIG. 22 is a block diagram showing a concept of the biological substance detection device 2 according to the present technology. The biological substance detection device 2 according to the present technology includes at least the above biological substance detection chip 1 according to the present technology and an analysis unit 21. In addition, according to their purpose, a light emission unit 22, a storage unit 23, a display unit 24, a temperature control unit 25 and the like can be provided. Hereinafter, respective units will be described. Here, since the biological substance detection chip 1 is as described above, descriptions thereof will be omitted here.

### (1) Analysis Unit 21

In the analysis unit 21, optical information acquired by the biological substance detection chip 1 is analyzed. For example, based on the optical information acquired by the biological substance detection chip 1, checking whether the biological substance S is present, checking whether there is an interaction with the biological substance S, and screening of medicinal components and the like are performed.

Here, the analysis unit 21 may be implemented in a personal computer or a CPU, or may be stored as a program in a hardware resource including a recording medium (for example, a nonvolatile memory (a USB memory), an HDD, or a CD) and the like, and can function by a personal computer or a CPU.

### (2) Light Emission Unit 22

The biological substance detection device 2 according to the present technology can include, for example, the light emission unit 22 for emitting excitation light. The light emission unit 22 emits light to the biological substance S held on the holding surface 111 of the biological substance detection chip 1. Here, in the biological substance detection device 2 according to the present technology, the light emission unit 22 is not essential, and light can be emitted to the biological substance S using an external light emission device or the like.

The type of light emitted from the light emission unit 22 is not particularly limited, but in order to reliably generate fluorescence or scattered light from microparticles, light having a constant light direction, wavelength, and light intensity is desirable. As an example, a laser, an LED and the like may be exemplified. When a laser is used, the type thereof is not particularly limited, and an argon ion (Ar) laser, a helium-neon (He—Ne) laser, a dye laser, a krypton (Cr) laser, a semiconductor laser, and a solid laser in which a semiconductor laser and a wavelength conversion optical element are combined can be used alone or two or more thereof can be freely used in combination.

According to their purpose, a plurality of light emission units 22 may be provided. For example, one light emission unit 22 may be provided for each pixel 11 of the biological substance detection chip 1. In addition, when a substrate in which light emitting elements such as LEDs are arranged at positions corresponding to the pixels 11 of the biological substance detection chip 1 is laminated on the biological substance detection chip 1, light can be emitted to the biological substance S.

### (3) Storage Unit 23

The biological substance detection device 2 according to the present technology can include the storage unit 23 in which various types of information are stored. The storage unit 23 can store all items related to detection such as optical data acquired by the biological substance detection chip 1, analysis data generated by the analysis unit 21, and optical data emitted by the light emission unit 22.

In the biological substance detection device 2 according to the present technology, the storage unit 23 is not essential, and an external storage device may be connected. As the storage unit 23, for example, a hard disk or the like can be used.

### (4) Display Unit 24

The biological substance detection device 2 according to the present technology can include the display unit 24 that displays various types of information. The display unit 24 can display all items related to detection such as optical data acquired by the biological substance detection chip 1, analysis data generated by the analysis unit 21, optical data emitted by the light emission unit 22, data stored in the storage unit 23 and the like.

In the biological substance detection device 2 according to the present technology, the display unit 24 is not essential, and an external display device may be connected. As the display unit 24, for example, a display, a printer or the like can be used.

### (5) Temperature Control Unit 25

The biological substance detection device 2 according to the present technology can include the temperature control unit 25 that keeps the biological substance S held on the holding surface 111 of the biological substance detection chip 1 at a predetermined temperature and heats or cools it to a predetermined temperature. For example, when the biological substance S is an enzyme, the temperature control unit 25 can control the temperature so that an optimal temperature is maintained. In addition, when the biological substance S is a nucleic acid, and the presence of hybridization is detected using the present technology, the temperature control unit 25 can perform control so that the temperature range in which hybridization is possible is maintained. As the temperature control unit 25, a thermoelectric element such as a Peltier element can be used.

According to their purpose, a plurality of temperature control units 25 may be provided. For example, one temperature control unit 25 may be provided for each pixel 11 of the biological substance detection chip 1. In addition, when a substrate in which thermoelectric elements are arranged at positions corresponding to the pixels 11 of the biological substance detection chip 1 is laminated on the biological substance detection chip 1, the temperature of the biological substance S can be controlled.

Here, in the biological substance detection device 2 according to the present technology, the temperature control unit 25 is not essential, and the temperature of the biological substance S can be controlled using an external temperature control device or the like.

### 4. Biological Substance Detection System 3

FIG. 27 is a block diagram showing a concept of the biological substance detection system 3 according to the present technology. The biological substance detection system 3 according to the present technology includes at least the above biological substance detection chip 1 according to the present technology and an analysis device 31. In addition, according to their purpose, a light emission device 32, a storage device 33, a display device 34, a temperature control device 35 and the like can be provided.

The biological substance detection chip 1 and respective devices can be connected via a wired or wireless network. Here, since details of respective devices are the same as details of respective units of the biological substance detection device 2 of the present technology described above, descriptions thereof will be omitted here.

Here, in the present technology, the following configurations can be used.

(1) A biological substance detection chip which is composed of a plurality of pixels in which the pixel at least includes a holding surface on which a biological substance is held and a photoelectric conversion unit that is provided below the holding surface and provided on a semiconductor substrate, wherein a partition wall made of a conductor is provided between the pixels on the holding surface.

(2) The biological substance detection chip according to (1), wherein a voltage is applied to the partition wall when the biological substance is detected.

(3) The biological substance detection chip according to (2), wherein a positive voltage or a negative voltage is applied to all of the partition walls.

(4) The biological substance detection chip according to (2), wherein a positive voltage or a negative voltage is applied to the partition wall.

(5) The biological substance detection chip according to any one of (2) to (4), wherein a magnitude of a voltage applied to the partition wall is able to be changed for each partition wall.

(6) The biological substance detection chip according to any one of (1) to (5), wherein some or all of the partition walls that are able to be conductive on the holding surface are covered with a protective film.

(7) The biological substance detection chip according to any one of (1) to (6), wherein the biological substance is one or more biological substances selected from among nucleic acids, proteins, cells, microorganisms, chromosomes, ribosomes, mitochondria, organelles (cell organelles), and complexes thereof.

(8) A biological substance detection device, including; a biological substance detection chip which is composed of a plurality of pixels in which the pixel includes at least a holding surface on which a biological substance is held and a photoelectric conversion unit that is provided below the holding surface, and in which a partition wall made of a conductor is provided between the pixels on the holding surface; and an analysis unit that analyzes electrical information acquired by the biological substance detection chip.

(9) A biological substance detection system, including; a biological substance detection chip which is composed of a plurality of pixels in which the pixel includes at least a holding surface on which a biological substance is held and a photoelectric conversion unit that is provided below the holding surface, and in which a partition wall made of a conductor is provided between the pixels on the holding surface; and an analysis device that analyzes electrical information acquired by the biological substance detection chip.

### REFERENCE SIGNS LIST

1 Biological substance detection chip

### 11 Pixel

S Biological substance
111 Holding surface
12 Semiconductor substrate
112 Photoelectric conversion unit
13 Partition wall

### 14 Insulator

### 15 Gate

113 P-type region
21 Analysis unit
22 Light emission unit
23 Storage unit
24 Display unit
25 Temperature control unit
31 Analysis device
32 Light emission device
33 Storage device
34 Display device
35 Temperature control device

1. A biological substance detection chip which is composed of a plurality of pixels in which the pixel at least includes a holding surface on which a biological substance is held and a photoelectric conversion unit that is provided below the holding surface and provided on a semiconductor substrate, wherein a partition wall made of a conductor is provided between the pixels on the holding surface.

2. The biological substance detection chip according to claim 1,
wherein a voltage is applied to the partition wall when the biological substance is detected.

3. The biological substance detection chip according to claim 2,
wherein a positive voltage or a negative voltage is applied to all of the partition walls.

4. The biological substance detection chip according to claim 2,
wherein a positive voltage or a negative voltage is applied to the partition wall.

5. The biological substance detection chip according to claim 2,
wherein a magnitude of a voltage applied to the partition wall is able to be changed for each partition wall.

6. The biological substance detection chip according to claim 1,
wherein some or all of the partition walls that are able to be conductive on the holding surface are covered with a protective film.

7. The biological substance detection chip according to claim 1,
wherein the biological substance is one or more biological substances selected from among nucleic acids, proteins, cells, microorganisms, chromosomes, ribosomes, mitochondria, organelles (cell organelles), and complexes thereof.

8. A biological substance detection device, comprising:
a biological substance detection chip which is composed of a plurality of pixels in which the pixel includes at least a holding surface on which a biological substance is held and a photoelectric conversion unit that is provided below the holding surface, and in which a partition wall made of a conductor is provided between the pixels on the holding surface; and
an analysis unit that analyzes electrical information acquired by the biological substance detection chip.

9. A biological substance detection system, comprising:
a biological substance detection chip which is composed of a plurality of pixels in which the pixel includes at least a holding surface on which a biological substance is held and a photoelectric conversion unit that is provided below the holding surface, and in which a partition wall made of a conductor is provided between the pixels on the holding surface; and
an analysis device that analyzes electrical information acquired by the biological substance detection chip.
